(19) United States
(12) Patent Application Publication
(10) Pub. No.: US 2018/0146898 A1
(43) Pub. Date: May 31, 2018

(54) SWEAT SENSING DEVICE CORTISOL MEASUREMENT

(71) Applicant: Eccrine Systems, Inc., Cincinnati, OH (US)

(72) Inventors: Gavi Begtrup, Cincinnati, OH (US); Robert Beech, Cincinnati, OH (US); Jason Heikenfeld, Cincinnati, OH (US)

(73) Assignee: Eccrine Systems, Inc., Cincinnati, OH (US)

(21) Appl. No.: 15/576,146

(22) PCT Filed: Jun. 6, 2016
(86) PCT No.: PCT/US16/35972

§ 371 Date: Nov. 21, 2017

Related U.S. Application Data
(60) Provisional application No. 62/171,578, filed on Jun. 5, 2015.
(60) Provisional application No. 62/180,698, filed on Jun. 17, 2015.
(60) Provisional application No. 62/336,982, filed on May 16, 2016.

(51) Int. Cl. A61B 5/145; A61B 5/00; A61B 5/01; A61B 5/024; A61B 5/11; A61B 5/16; A61B 10/00

(52) U.S. Cl. CPC A61B 5/14521; A61B 5/4809; A61B 5/14546; A61B 5/01; A61B 5/02405; A61B 5/1116; G01N 33/487; A61B 5/165; A61B 5/4266; A61B 5/14539; A61B 10/0064; A61B 5/7285; A61B 5/6833

(57) ABSTRACT

The present disclosure includes a device and method of measuring an individual's cortisol awakening response; a device and method of measuring an individual's diurnal cortisone level, including the basal cortisol level; a device and method of indicating an individual's stress profile based on sweat cortisol measurements; a device for measuring and interpreting sweat analytes relevant to risk-taking behavior; and a method of determining an individual's risk-taking propensity based on measurement and development of sweat analyte profiles.

### CROSS-REFERENCE TO RELATED APPLICATIONS

The present application has specification that builds upon U.S. Provisional Application No. 62/171,578, filed Jun. 5, 2015, U.S. Provisional Application No. 62/180,698, filed Jun. 17, 2015, and U.S. Provisional Application No. 62/336,982, filed May 16, 2016, the disclosures of which are hereby incorporated herein by reference in their entirety.

### BACKGROUND OF THE INVENTION

Sweat sensing technologies have enormous potential for applications ranging from athletics, to neonatology, to pharmacological monitoring, to personal digital health, to name a few applications. This is because sweat contains many of the same biomarkers, chemicals, or solutes that are carried in blood, which can provide significant information which enables one to diagnose ailments, health status, toxins, performance, and other physiological attributes even in advance of any physical sign. Furthermore sweat itself, and the action of sweating, or other parameters, attributes, solutes, or features on or near skin or beneath the skin, can be measured to further reveal physiological information.

In particular, sweat sensing devices hold tremendous promise for use in workplace safety, athletic, military, and clinical diagnostic settings. A primary goal of the present invention is to provide decision support to a device user that is informative at the level of the individual patient. A sweat sensing patch worn on the skin and connected to a computer network via a reader device, such as a smart phone or other portable or stationary device, could aid in recognition of the physiological state of an individual, and relay crucial data about dehydration levels, physiological stress levels, ovulation cycle or other physiological states. In certain settings, sweat sensors may continuously monitor certain aspects of an individual's physiological state and communicate relevant information to a reader device or computer network, which would then compare collected data to threshold readings and generate notification messages to the individual, a caregiver, a work supervisor, or other device user.

In particular, an individual's cortisol profile may be indicated by monitoring cortisol levels as they present in sweat. Methods to derive meaningful data about an individual's cortisol levels using a sweat sensing device, and thereby to contribute information about the individual's physical and mental health, are contemplated within the scope of the present invention.

Before continuing with the background, a variety of definitions should be made, these definitions gaining further appreciation and scope in the detailed description and embodiments of the present invention.

Sweat sensor means any type of sensor that measures a state, presence, flow rate, solute concentration, solute presence, in absolute, relative, trending, or other ways in sweat. Sweat sensors can include, for example, potentiometric, amperometric, impedance, optical, mechanical, antibody, peptide, aptamer, or other means known by those skilled in the art of sensing or biosensing.

Sweat sensor data means all of the information collected by device sensor(s) and communicated via the device to a user or a data aggregation location.

Correlated aggregated sweat sensor data means sweat sensor data that has been collected in a data aggregation location and correlated with outside information such as time, temperature, weather, location, user profile, other sweat sensor data, or any other relevant data.

Cortisol awakening response (“CAR”) means the surge in blood cortisol levels, typically a 50% increase over diurnal levels, that occurs shortly after an individual wakes after an extended sleep.

Diurnal cortisol level means the normal cyclic variation in blood cortisol that is generally correlated to an individual's circadian sleep-wake cycle.

Baseline cortisol profile means a value developed for an individual, a phenotype, or a relevant population (such as individuals of a certain age, weight, occupation, etc.) that reflects a long term diurnal cortisol levels that are compared to a daily or short term basal, or peak cortisol measurement by a sweat sensing device. For example, measurements of an individual's basal and peak cortisol levels taken over several days and averaged could serve as a baseline cortisol profile for that individual.

Basal testosterone level means the lowest sweat testosterone concentration measured on an individual over a circadian testosterone cycle.

Peak testosterone level means the highest sweat testosterone concentration measured on an individual over a circadian testosterone cycle.

Cortisol reactivity means the magnitude of an individual's cortisol response to a stress stimulus as compared to a basal cortisol value.

EAB sensor means an electronic aptamer-based sensor, such as is disclosed in U.S. Pat. Nos. 7,803,542 and 8,003,374.

This has served as a background for the present invention, including background technical invention needed to fully appreciate the present invention, which will now be summarized.

### SUMMARY OF THE INVENTION

The present invention is premised on the realization that sweat can be effectively analyzed in a single, continuous, or repeated manner inside the same device, and addresses applications of a device based on such capabilities. Specifically, the present invention provides: a method of measuring an individual's cortisol awakening response; a method of measuring an individual's diurnal cortisone level, including the basal cortisol level; a method of indicating an individual's stress profile based on sweat cortisol measurements; a device for measuring and interpreting sweat analytes relevant to risk taking behavior; and a method of determining an individual's risk taking propensity based on measurement and development of sweat analyte profiles.

### BRIEF DESCRIPTION OF THE DRAWINGS

The objects and advantages of the present invention will be further appreciated in light of the following detailed descriptions and drawings in which:

FIG. 1 is an example sweat sensing device of the present disclosure.

FIG. 2 is a more detailed depiction of a sweat sensing device of the present disclosure.

FIG. 3 is an example diagram of an individual's cortisol awakening response.

FIG. 4 is an example diagram of an individual's diurnal cortisol level.

FIG. 5 is an example flow chart representing a method of indicating an individual's stress profile.

FIG. 6 is an example chart representing a method by which the disclosed invention may determine a wearer's risk-taking propensity.

### DETAILED DESCRIPTION OF THE INVENTION

The detailed description of the present invention will be primarily, but not entirely, limited to methods and sub-methods using wearable sweat sensing devices. Therefore, although not described in detail here, other essential steps which are readily interpreted from or incorporated along with the present invention shall be included as part of the present invention. The specification for the present invention provides specific examples to portray inventive steps, but which will not necessarily cover all possible embodiments commonly known to those skilled in the art. For example, the specific invention will not necessarily include all obvious features needed for operation. Several specific, but non-limiting, examples can be provided as follows. The invention includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as PCT/US2013/035092, PCT/US2015/55756; U.S. Provisional Application No. 62/180,698, and U.S. Provisional Application No. 62/171,578, U.S. Provisional Application No. 62/336,982, each of which is included herein by reference in their entirety. Many of the auxiliary features of the method may, or may not, also require aspects of the sweat sensing device. It is understood that many such sensors require two or more electrodes, reference electrodes, or additional supporting technology or features, which are not captured in the description herein, such as body and sweat temperature sensors, and sweat pH sensors.

With reference to FIG. 1, a representative sweat sensor device 100 to which the present disclosure applies is placed on or near skin 12. The sweat sensor device may be fluidically connected to skin or regions near skin through microfluidics or other suitable techniques. Device 100 is in wired communication 152 or wireless communication 154 with a reader device 150, which could be a smart phone or portable electronic device, or for some devices, the device 100 and reader device 150 can be combined. Communication 152 or 154 is not constant and could be a simple one-time data download from device 100 once it has completed its measurements of sweat.

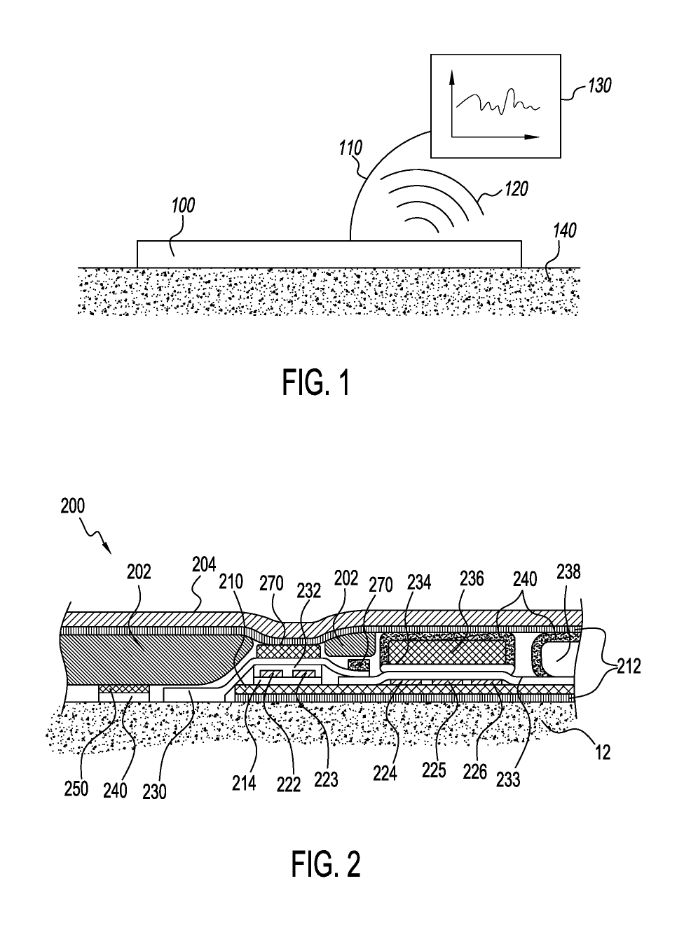

With reference to FIG. 2, a more detailed partial view of a sweat sensing device is provided as device 200. The device 200 may include the following elements: a filler material 202; a textile covering 204; a substrate 210; adhesive 212; a self-leveling material 214; a wicking volume reducing component 230; a sweat stimulant gel composed of sweat stimulant and agar 240; and an iontophoresis electrode 250.

The device further includes at least one sensor(s) 222, 223; an optional hydrogel 232 for enhancing fluidic contact between substrate 210 and a rigid component 270 such that the wicking volume reducing component 230 and the at least one sensor 222, 223 are in fluidic contact at all times. The device further includes a wicking or microfluidic component 233; at least one electrochemical aptamer sensor(s) 224, 225, 226; a forward osmosis membrane 234; a polymer seal 214; an osmosis pumping material 236; and a wicking pumping material 238.

With further reference to FIG. 2, the device could operate as follows: electrode 250, and gel 240 provide iontophoretic sweat stimulation as needed. Once sweat is stimulated, electrode 250 can also be used to measure skin impedance, which can be used to derive a proportional measure of electroporation and/or sweat generation rate. Wicking volume reducing component 230 then wicks stimulated sweat off the skin surface and carries the sweat sample to at least one sensor(s) 222, 223, which would measure Na+, K+, and pH. Wicking component 230 may also include a thermal flow meter sensor to measure sweat rate. Wicking component 233 then transports the sweat sample onto electrochemical aptamer sensors 224, 225, 226. The analytes will be concentrated as water and small sweat solutes are transported through the forward osmosis membrane 234 and out of the sweat sample. Finally, wicking material 238 absorbs the sweat sample, and at least partially drives sweat sample flow through the device.

As disclosed, a sweat sensing device might include a plurality of sensors to improve detection of sweat analytes, including a reference electrode, a pH sensor, a temperature sensor, a plurality of galvanic skin response sensors, sweat conductivity sensors, a skin impedance sensor, a capacitive skin proximity sensor, and an accelerometer. Many of the auxiliary features of the invention may, or may not, require other aspects of a sweat sensing device, including two or more counter electrodes, reference electrodes, or additional supporting technology or features, which are not captured in the description herein, such as body or sweat temperature and pH sensors, an onboard real-time clock, onboard flash memory (i.e. 1 MB minimum), Bluetooth™ or other communications hardware, and a multiplexer to process a plurality of sensor outputs.

The disclosed sweat sensor device also includes computing and data storage capability sufficient to operate the device, which incorporates the ability to conduct communication among system components, to perform data aggregation, and to execute algorithms capable of generating notification messages. The device may have varying degrees of onboard computing capability (i.e., processing and data storage capacity). For example, all computing resources could be located onboard the device, or some computing resources could be located on a disposable portion of the device and additional processing capability located on a reusable portion of the device. Alternatively, the device may rely on portable, fixed or cloud-based computing resources.

The sweat sensing device's data aggregation capability may include collecting all of the sweat sensor data generated by sweat sensor devices and communicated to the device. The aggregated sweat sensor data could be de-identified from individual wearers, or could remain associated with an individual wearer. Such data can also be correlated with outside information, such as the time, date, medications, drug sensitivity, medical condition, activity performed by the individual, motion level, fitness level, mental and physical performance during the data collection, body orientation, the proximity to significant health events or stressors, age, sex, health history, or other relevant information. The reader device or companion transceiver can also be configured to correlate speed, location, temperature or other relevant data with the sweat sensor data. The data collected could be made accessible via secure website portal to allow sweat system users to perform safety, compliance and/or care monitoring of target individuals. The sweat sensor data monitored by the user includes real-time data, trend data, or may also include aggregated sweat sensor data drawn from the system database and correlated to a particular user, a user profile (such as age, sex or fitness level), weather condition, activity, combined analyte profile, or other relevant metric. Trend data, such as a target individual's hydration level over time, could be used to predict future performance, or the likelihood of an impending physiological event. Such predictive capability can be enhanced by using correlated aggregated data, which would allow the user to compare an individual's historical analyte and external data profiles to a real-time situation as it progresses, or even to compare thousands of similar analyte and external data profiles from other individuals to the real-time situation. Sweat sensor data may also be used to identify wearers that are in need of additional monitoring or instruction, such as the need to drink additional water, or to adhere to a drug regimen.

Because the sweat sensor device could produce potentially sensitive physiological data, some database fields will be routinely encrypted. A preferred encryption method is the Advanced Encryption Standard. The device will access a random 128 bit encryption and decryption key that will be generated and stored by a companion reader device when needed for data transmission. In addition, because some sweat sensor data may repeat frequently, additional protection will be provided by introducing a random initialization vector before the encryption of each value. This will prevent observable patterns from emerging in the encrypted sweat sensor data. Other encryption methods and steps may be required and will be applied according to best practices, as known to those skilled in the art.

Sweat is known to contain a large number of compounds that may be used to indicate an individual's physiological state. In general, determining an individual's physiological state is a significant challenge. Not only is every individual different in terms of how a particular physiological state may present, but even a simple physiological state or disorder is a complex set of biological processes that does not readily lend itself to reduction. For example, the state of hydration, while seemingly a familiar concept, is quite difficult to describe from a medical standpoint, and is susceptible to distinct definitions (i.e., loss of blood plasma volume, versus loss of body water content). Consequently, a definitive diagnosis of a physiological condition often is not possible. Rather, it becomes necessary to divide individuals according to phenotypes or susceptibilities that indicate a certain way a physiological state is likely to manifest in those individuals.

These phenotypes may be indicated by analyte signatures that emerge in sweat. Among the most common substances found in sweat are the following: Na⁺, Cl⁻, K⁺, Ammonium (NH₄⁺), urea, lactate, glucose, serine, glycerol, cortisol, and pyruvate. It will likely be necessary to build data across multiple individuals correlating physiological states with sweat sensor readings. By this means, certain phenotypes may be identified for which a given physiological state will manifest in a discernible sweat analyte signature. Further, translation of analyte concentrations and ratios to meaningful physiological information will have to account for a number of variabilities unrelated to differences in concentrations. For example, sweat concentrations of analytes relative to blood or plasma concentrations are known to vary depending on sweat rate, the body location from which a sample is taken, kidney or liver disease or function, external temperatures, and other factors. To develop meaningful physiological information, it may therefore be necessary to employ algorithms and techniques that reflect how the various analyte signatures change in response to these variabilities.

Of the common sweat analytes cited above, cortisol is particularly relevant for indicating a number of physical and mental conditions, and behaviors. Cortisol is a steroid hormone synthesized in the adrenal glands upon the pituitary gland's release of adrenocorticotropic hormone. Cortisol has many functions in the body, including the facilitation of glucose metabolism, regulation of electrolytes and water balance, the management of stress, and mediation of immune response. Because of its importance, imbalances in cortisol levels can have profound effects on the human body.

For example, cortisol is crucial to the body's management of stress. Under normal circumstances, cortisol prepares the organism to respond to external circumstances by stimulating glucose production, increasing blood pressure, and reducing swelling and pain. Cortisol's role in an individual's risk-taking behavior is related to this stress management function. Under exposure to short-duration stressors, cortisol prepares the organism to respond to external circumstances by activating the body's sympathetic nervous system, the so-called “fight or flight” mode. Under exposure to long-term or chronic stress, cortisol levels can remain elevated for extended periods. Because of its close relationship to alert and resting states, cortisol variations due to stress and other causes can also disrupt sleep cycles, and in turn, sleep deprivation or sleep apnea may increase blood cortisol levels. Cortisol's normal function in metabolism is to counteract insulin and promote insulin resistance, thereby creating a temporary hyperglycemic state. If this hyperglycemic state remains in place due to chronically high cortisol levels, diabetes can result. Cortisol normally acts to regulate bone and connective tissue formation by inhibiting collagen production, transporting potassium out of cells and inhibiting the uptake of calcium by the small intestine. In excessive amounts, therefore, cortisol promotes osteoporosis and hypokalemia. Cortisol's role in maintaining the body's water/electrolyte balance is to promote sweating, diuresis, sodium retention, and potassium excretion. Along with its vaso-constrictive effects, excessive cortisol may therefore lead to hypernatremia, increased high blood pressure and cardiovascular disease. Cortisol is also instrumental in human immune response, acting to suppress cell-mediated immune response, thereby reducing inflammation, and activating humoral immunity, which promotes the mobilization of antibodies. In excess amounts, cortisol can degrade effective immune response, and slow wound healing. Cortisol is also thought to promote the formation of short-term “flash” memory for threat avoidance, but in excess may impair learning and memory recall.

Excess cortisol levels may be caused by chronic stress levels, pituitary or adrenal disorder, kidney or liver disease, excessive use of cortisol-like drugs, and tumors. Conversely, deficient cortisol levels may be caused by pituitary or adrenal disorder, steroid use, autoimmune disorders, and tumors. Deficient cortisol may manifest in hypoglycemia, dehydration, low blood pressure, and other conditions.

Interpreting the physiological meaning of cortisol levels as they are detected in sweat is a considerable challenge because of cortisol's normal variation cycles, and because external events, individual health, and phenotypical characteristics can considerably alter measured cortisol levels. As a first order assessment of sweat cortisol, therefore, the device should be configured to assess normal cortisol variability. Cortisol experiences two daily cycles of variability: the diurnal cortisol level and cortisol awakening response.

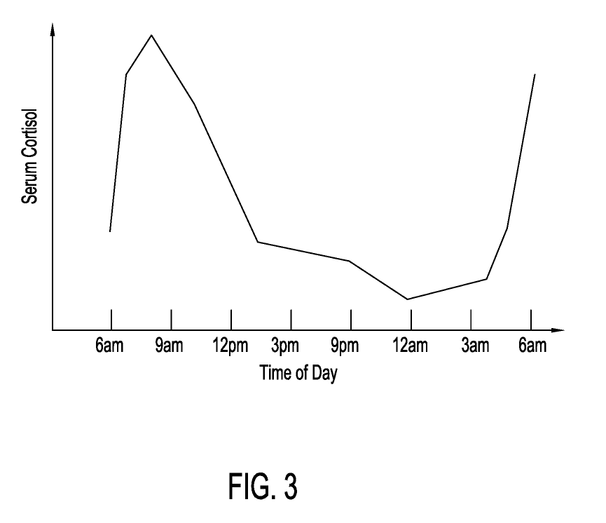

With reference to FIG. 3, diurnal cortisol levels are closely tied to an individual's sleep cycle. Cortisol production and corresponding cortisol blood levels increase during the second half of an extended sleep (as opposed to a nap), peaking in the early morning. Thereafter, diurnal cortisol levels generally decrease during the waking hours, reaching a nadir, or basal cortisol level, a couple of hours after sleep begins. These blood cortisol levels are generally in the range of 140 to 700 Mol/L in the morning, and 80 to 350 nMol/L at midnight (assuming a night sleep cycle). Diurnal cortisol levels may be significantly affected by the various disorders or chronic conditions affecting cortisol levels generally, but may also vary in response to shorter term circumstances, such as physical or emotional stress, pregnancy, hypoglycemia, food or water intake prior to testing, or taking a course of steroids or corticoids, birth control pills or estrogen.

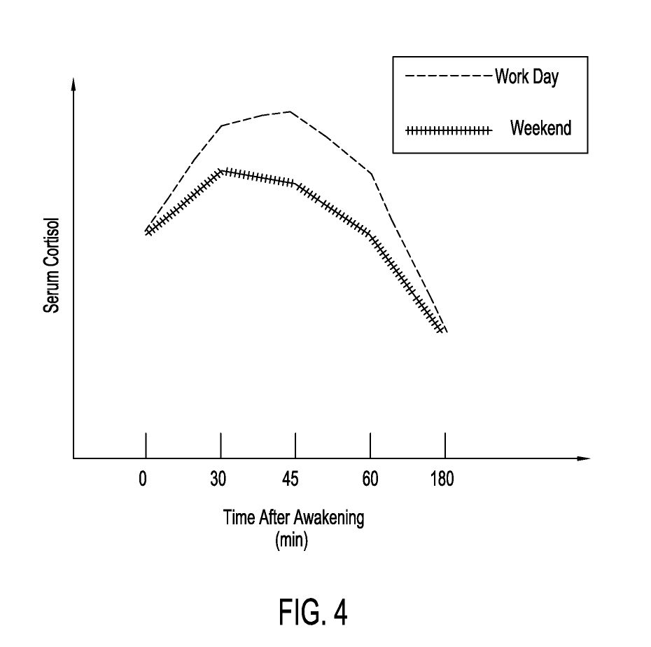

Cortisol awakening response is generally more predictable, and hence potentially more meaningful, than diurnal cortisol level. With reference to FIG. 4, CAR is a sharp increase of about 50% over the then-current diurnal cortisol level, and peaks in blood roughly thirty minutes after an individual wakes from a night's sleep. The CAR is thought to play a role in activating the body's hypothalamic-pituitary-adrenal (HPA) axis to orient the organism and prepare for the day's demands. CAR is specifically linked to the awakening event, and its magnitude is independent of diurnal cortisol variation. Assessment of an individual's CAR profile may therefore indicate that individual's capacity to activate the HPA axis, and in turn to respond effectively to stressors. An individual's typical CAR value is genetically determined, and remains fairly stable day-to-day, absent short-term factors that are known to affect CAR levels.

An individual's CAR response may be increased or decreased by various factors. CAR response is increased by waking earlier in the morning, waking in light as opposed to darkness, awaking for work or an athletic contest as opposed to leisure, having lower socioeconomic status or higher short-term stress. CAR may be reduced, i.e., display a flatter profile, in individuals getting poor sleep or sleep with excess environmental noise, individuals experiencing chronic stress, suffering from pain, or suffering from physical overexertion or over-training. An accurate sweat measurement of CAR, therefore, would indicate an individual's short-term response to physical exertion, mental stress, and sleep. Further, because it is less dependent than the diurnal cortisol profile on longer-terms factors such as physical fitness, chronic stress, depression and disease, CAR has emerged as the single most informative measure of cortisol in humans.

One embodiment of the present invention is a method to use a sweat sensing device to monitor cortisol awakening response. Because CAR is associated with awakening after an extended (i.e. more than 4 hours) sleep, the user should apply the device before the individual goes to sleep. To properly record data on the entire CAR, the device should begin taking readings as the individual wakes, and continue periodic readings at chronologically assured sampling rates for approximately 45 minutes. The device may be configured to determine that the wearer has awakened by several means known in the art. These may include programming the device with a planned wake time, or synchronizing the device with an alarm clock, a smartphone application, transceiver, or other computing device capable of communication with the device. Such methods will need to distinguish between planned wake times and actual wake times, for example by distinguishing between when someone has turned off the alarm, or merely hit the snooze function.

Alternatively, and preferentially, the sweat sensing device may be configured to operate in conjunction with sensors capable of determining that the wearer is awakening, such as a sleep monitor, a pulse oximetry sensor, a heart rate monitor, accelerometers, or other means to measure circulatory activity, posture and movement. Such sensors are commonly available as part of wearable fitness tracker products. Once activated, the device will ideally take at least one valid reading near the time the wearer awakes to record a baseline value for the CAR. At least one additional valid reading should be taken near the 30 minute point after the wearer awakes, and at least one additional valid reading should be taken after the 30 minute point to verify the post-peak cortisol decline.

Unlike other sweat sensor applications, in which a sweat sensor reading may be timed to coincide with a desired sweat rate or sweat volume, CAR monitoring requires the valid sweat readings to be coordinated with a specific external event, namely, awakening. To capture the desired valid readings, therefore, the device must be able to stimulate sweat so that adequate sweat rate or volume, and thus chronologically assured sweat readings, may be timed to coincide with awakening and the subsequent CAR peak and decline.

Sweat stimulation for this application may be accomplished by chemical or electrical stimulation, such as iontophoretically-delivered carbachol, pilocarpine or methacholine, or it may be stimulated thermally via infrared pulse, or other heat source, such as a chemical heating pad. To achieve the proper timing, sweat stimulation must account for two basic factors, the individual's sweat response timing, and the required sweat volume of the device. An individual's sweat response timing is primarily dependent upon the difference between the individual's body temperature and sweat threshold. When a person is at rest, they generally have a relatively low body temperature, therefore significant sweat stimulation may be required to achieve the desired sweat rate and volume. It could therefore take up to several minutes or more to cause the person to begin sweating. Once sweating has begun, the person must generate enough sweat volume for the particular sweat sensor to take a chronologically assured reading. The timing for this factor is a function of sweat rate and the sweat sensor dead volume. Accounting for sweat response and chronological assurance, therefore, the sweat sensor should be able to take its first valid reading within several minutes of activation.

Similarly for capturing a measurement of the CAR peak, sweat stimulation should be coordinated to provide at least one valid reading near the 30 minute point after the wearer wakes, and another within a few minutes to record the CAR decline. In another embodiment of the invention, the sweat sensing device may be configured to increase sampling rate near the peak time to acquire a number of chronologically assured readings before and after the peak. The timing of peak sampling may be refined by accounting for the wearer's individual variability, which may be made available to the device in the form of correlated external data. External data may also be used to enhance the device measurement of the CAR profile by including information about the device wearer that may be relevant to the CAR value, such as the wearer's historical CAR profile, and the presence of known factors that may affect the CAR profile, such as recent physical exertion, fitness level, stress level, sleep patterns, or other relevant data.

In addition to measuring cortisol awakening response, in other embodiments of the invention, the device could be configured to measure the diurnal cortisol profile for an individual. The challenges of taking a meaningful diurnal cortisol profile measurement with a sweat sensing device are similar to those confronted when taking a CAR reading, but are amplified by the duration of monitoring required. Because of this, the diurnal cortisol cycle may be divided into three priorities: the basal cortisol level, the peak cortisol level, and the post-peak cortisol decline. With further reference to FIG. 3, a typical diurnal cortisol profile is anchored at the basal cortisol level, which usually occurs within a couple of hours after an individual normally goes to sleep, here shown at about 12:00 A.M. The cortisol level remains low until about three hours before the normal waking time, when it begins a sharp increase toward the peak level, which occurs in the morning, here shown at about 8:00 A.M. The cortisol level then begins a decline toward the basal value for the next day. Based on the priorities, a quality basal cortisol measurement would be given priority in terms of sensor power resources, sweat stimulation chemical supplies, and sensor longevity. Similarly, the cortisol peak reading would be given second priority in terms of device resources, and the device would use its remaining resources to monitor the post-peak decline. Preferably the entire cycle would be monitored by the same device, but if necessary, additional patches could be used to cover the cycle.

In one embodiment of the invention, the device user could apply the patch at bedtime. The device may mark the start of sleep by activation upon patch application, or alternatively, could incorporate or be in communication with other sensors, such as sleep monitors, heart monitors, pulse oximetry, temperature or accelerometers to determine when the wearer began sleep. The device would be configured to start sampling for the basal cortisol level at 1 hour and 30 minutes after sleep began. In another embodiment, the device may be configured to begin sampling at a particular time, such as midnight, which, for example is known to be an hour and a half past the wearer's normal sleep time. To capture the basal cortisol level, the device would be configured to take at least one, and preferably multiple, readings. As with CAR sampling discussed above, the device may need to stimulate sweat in order to ensure chronologically assured readings during the window. In some embodiments, the sweat sensing device may stimulate sweat to take chronologically assured readings every few minutes, for example every 15 minutes, for a set window, such as an hour. In other embodiments, the device may stimulate sweat readings every few minutes until the cortisol level passes the basal number and definitively begins to increase. In some embodiments, the device may monitor, or incorporate other sensors that monitor, skin or body temperature, sweat onset temperature, and sweat rate.

The secondary priority for diurnal cortisol monitoring is capturing the peak cortisol level, which occurs approximately 2 hours after waking. A similar process to the basal cortisol level may be pursued to capture the peak cortisol level. In this case, the sweat sensing device would be configured to begin taking readings one and a half hours after the individual awakes, or the device could be configured to operate at a set time, such as 9:00 A.M., that is an hour and a half past the individual's normal wake time. The device would continue to stimulate sweat (if necessary) and take chronologically assured readings for a certain amount of time, for example an hour, or in other embodiments, could continue to take sweat readings until the cortisol levels passed the peak, and definitively began to decline.

The tertiary priority for the sweat sensing device is to monitor the post-peak cortisol decline. During this portion of the cycle, the device would be configured to stimulate sweat (if necessary), and take periodic chronologically assured sweat readings, for example, every hour. In some embodiments, the device may be configured to increase its sampling rate in response to unexpected increases in the detected cortisol level, or in response to other relevant information, for example, a change in heart rate, skin or body temperature, sweat rate, sweat onset temperature, the consumption of a meal, a stress event, etc. The device could thus be used to monitor near real time changes in cortisol levels in response to short-term factors.

Unlike CAR, which is typically a predictable percentage increase over basal cortisol levels, diurnal cortisol values may vary widely from individual to individual based on long term factors such as fitness level, chronic stress, depression, medication, disease, among others. Therefore, in order to attain a diurnal cortisol measurement that provides meaningful information about the effects of short-term factors, such as sleep loss or overexertion, an individual's baseline cortisol profile may be developed. In some embodiments of the present invention, this baseline cortisol profile may be estimated based on relevant information about the individual, such as the person's age, weight, sleep habits, physical fitness, stress level, medications, etc. The individual's information may then be compared to aggregated data about other individuals in a database, and a baseline cortisol profile from comparable individuals would be selected. In other embodiments, the individual's diurnal cortisol may be monitored over a number of days, perhaps under controlled conditions, to develop a baseline cortisol profile for that individual. Once the baseline cortisol profile is developed, short-term variations in diurnal cortisol can be distinguished and analyzed. For example, an individual's basal cortisol will often be elevated over normal levels after the individual experiences a significant stress event. With the baseline cortisol profile in hand, such variations may be readily appreciated.

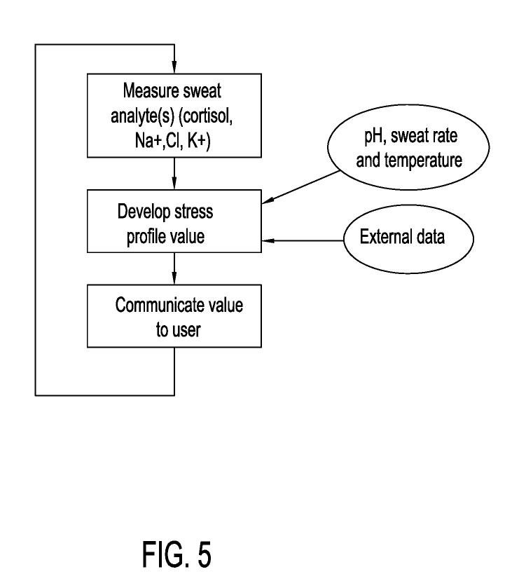

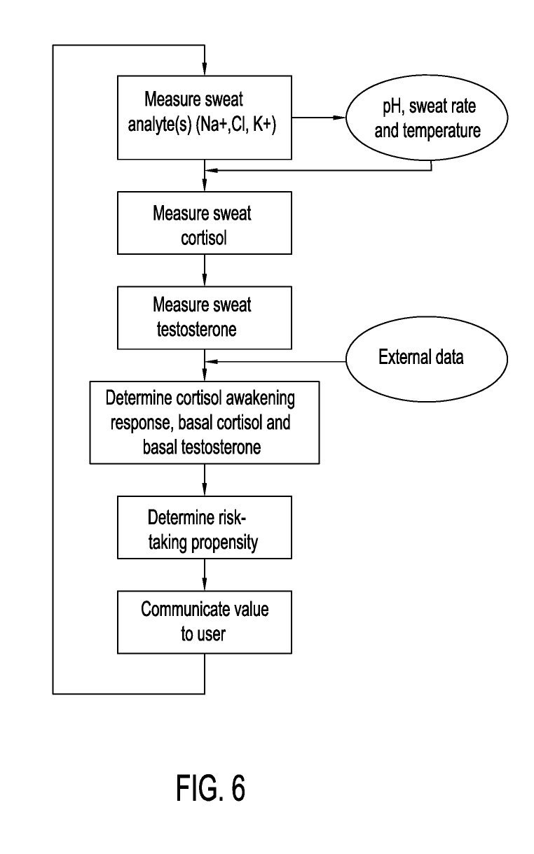

With reference to FIG. 5, in another embodiment of the invention, the sweat sensing device is configured and used to indicate (1) whether an individual is experiencing a significant discrete stress event, such as a panic attack, or (2) chronically occurring stress events. When an individual experiences stress, the body responds by releasing a series of hormones. These include adrenocorticotropic hormone, which triggers the release of norepinephrine, epinephrine, and cortisol from the adrenal glands, as well as dopamine and serotonin from nerve cells. Estrogen and testosterone may also affect the manifestation of stress in an individual. The stress response can be characterized by the general adaptation syndrome (“GAS”), which includes an initial alert phase, a resistance phase and a recovery or exhaustion phase. During the alert phase, blood Na+, Cl—, and glucose levels drop, and sweat rate increases or there is a sudden onset of sweating. Then, as cortisol and other hormones take effect, glucose, lipid and amino acid levels increase, and sweating diminishes. During the recovery/exhaustion phase, sweating may return.

The individual's stress-related sweat data may be used to develop a stress profile value. By comparing sweat concentrations and ratios of cortisol and other relevant analytes as well as sweat onset rate and sweat rate, the stress profile would reflect, with reasonable accuracy, whether a person was likely experiencing a panic attack, or a less severe stress response. Further, the stress profile could be monitored over time to determine whether the wearer experienced frequent stress responses, and thus was subject to chronic stress. Chronic stress as indicated by high cortisol levels in urine is routinely used to predict whether an individual is at risk for developing conditions such as diabetes, osteoporosis, and cardiovascular disease. Therefore, the use of a stress profile as disclosed herein may prove useful in diagnosing such risk factors using sweat sensor data.

In another embodiment of the present disclosure, short-term and long-term stress has been shown to affect an individual's capacity for risk-taking. Acute short-term stress results in a cortisol response, or spike, and also increases overall cortisol production. Both the magnitude of the initial cortisol response, and the amount of cortisol that is produced affect behavior following a stress event, which is generally characterized by increased fear and sensation-seeking. However, the propensity for risk-taking decreases with the amount of reactivity of an individual's cortisol levels, i.e., the larger the cortisol response to an acute stress event, the more inhibited a person's behavior will tend to be. Studies involving financial traders have shown that for females, reaction to short-term acute stress causes a relatively large cortisol spike, which in turn results in more cautious behaviors, or decreased propensity for risk-taking. See Ceccato, S., et al., “Increased risk taking in response to chronic stress in adults,” Frontiers in Psychology, Jan. 29, 2016. For males, however, short-term acute stress initiates a relatively muted cortisol spike, and is correlated with the individuals making more individual trades than usual, and with those trades showing greater deviation between prices and fundamental values. See Cueva, C., et al. In other words, short-term acute stress causes male financial traders in particular to engage in riskier trading behavior.

When chronic stress is factored into the equation, however, the sex differences tend to become less pronounced. Individuals of both sexes under chronic stress show a higher propensity for risk-taking when faced with short-term acute stress events. See Ceccato, S., et al., But see Kandasamy, N., et al., “Cortisol shifts financial risk preferences,” PNAS, Mar. 4, 2014, Vol. 111, No. 9 (finding decreased risk taking behaviors for people experiencing chronic stress). Such convergence in risk taking propensity for individuals of different sex may be attributable to the fact that chronic stress tends to reduce reactivity to short-term acute stress events for both males and females.

Because propensity for risk-taking behavior shows sex dependence, testosterone is a logical secondary sweat analyte that may improve the predictive value of a sweat sensing device for this application. Testosterone is an anabolic steroid and primary male sex hormone that, for mammals, is mainly produced in the male testes and in the female ovaries. The chief determinant in testosterone levels from individual to individual is sex. Male blood testosterone levels are typically 7 to 8 times higher than those of females. Testosterone plays a role in stress response by regulating acute stress response through HPA regulation. Higher basal levels of testosterone (relative to other members of the same sex) promote increased competitive and status-seeking behaviors, especially during periods of instability, i.e., during short-term stress events. Mehta, P., et al., “The social endocrinology of dominance,” J. of Personality & Social Psychology, 2008, Vol. 94, No. 6, 1078-1093.

Testosterone and cortisol interact to create profound effects on certain risk-taking behaviors. Individuals with high basal levels of testosterone (relative to other members of the same sex) tend to produce lower levels of cortisol in response to a social victory, and higher cortisol levels in response to social defeat. By contrast, individuals with low basal testosterone tended to respond with slightly lower cortisol production whether experiencing victory or defeat. Mehta, P., et al. Consequently, individuals with high basal testosterone may seek out situations leading to social victory, while avoiding situations leading to social defeat. Applied to financial trading, high basal testosterone may encourage risk taking behaviors in order to avoid a social defeat, such as might be associated with an individual having a negative trading day. In addition, high basal testosterone levels are correlated with increased risk-taking behavior when basal cortisol levels are low, but high testosterone individuals show less risk propensity when basal cortisol levels are high. Mehta, P., et al., “Testosterone and cortisol jointly modulate risk-taking,” J. of Phychoneuroendocrinology, March 2015. This relationship between testosterone and cortisol reinforces the chiefly male tendency to react to short-term acute stress with increased risk-taking, while chronic stress tends to mute the difference between the sexes in their responses to acute stress events.

Determining basal testosterone with a sweat sensing device requires similar techniques as used to detect cortisol. Since it is a hormone, an EAB sensor modality will be preferable, though amperometric or immunoassay sensors may also be used. Like cortisol, testosterone shows circadian variation, reaching its lowest level at night, and peaking in the morning. For males 30-40 years old, this variation manifests as a 20-25% decrease from morning to night. This difference becomes less pronounced with age, for example at age 70, diurnal variation is only about 10%. On average, younger men have about 17.7 nMol/L serum testosterone, while the figure for older men is about 12.1 nMol/L. Plymate, S., et al., “Circadian variation in testosterone,” J. of Andrology, September-October 1989. Because of the circadian variability in testosterone, it will be necessary to measure the nadir in testosterone, or the basal testosterone level in the evening, while an individual's peak testosterone level may be measured in the morning. To ensure sweat testosterone measurement at the proper time, similar techniques to those used in cortisol measurement, for example sweat stimulation and dynamic sensor activation and management, may be necessary to capture valid readings of the basal and peak testosterone levels. In addition, because basal testosterone levels may increase in anticipation of a short-term acute stress event, basal and or peak testosterone may need to be measured during a control period, or a profile may be developed for an individual, or for a particular phenotype of individual.

It may also be necessary to take a testosterone reading contemporaneously with another event, such as a short-term acute stress event experienced by a device wearer, or a cortisol reading taken by the device. For each example listed above, the timing of the sweat testosterone measurement may depend on a number of factors, including without limitation, any lag time between serum testosterone levels and correlated sweat concentrations, chronological assurance, time required for sample concentration, etc.

Therefore, in certain embodiments, the sweat sensing device disclosed herein may be configured to measure a wearer's sweat cortisol and testosterone levels to determine the wearer's propensity for risk-taking behavior. An initial step may include the determination of basal cortisol, basal testosterone, and cortisol awakening response. The measurements would establish a risk-taking assessment of a wearer's propensity for risk taking behavior. For example, elevated basal cortisol levels and a muted CAR would indicate that the wearer has been subject to chronic stress. The existence of chronic stress indicates an increased propensity for risk-taking behavior, regardless of sex or testosterone levels. As another example, the wearer may register a low basal cortisol level, and a large CAR response. Coupled with low basal testosterone, the wearer would have a decreased propensity for risk-taking because they would tend to have high cortisol reactivity to acute stress, and the low testosterone would augment the tendency to engage in risk avoidance behaviors.

In addition to a risk-taking assessment, the sweat sensing device may also be configured to analyze a wearer's cortisol reactivity to short-term stress events, and thereby calculate a risk-taking propensity. The particular difficulty for this type of sensing application is the need to have finely granular readings of cortisol, and in some cases, testosterone. An ideal cortisol reactivity measurement of a wearer's response to a stress event would include a measurement taken just prior to the event to assess the pre-event cortisol level. A second measurement would take place at the peak of the cortisol response to assess the magnitude of the cortisol response. A third, and perhaps subsequent measurements, would measure post-event cortisol levels to assess the increase in cortisol production resulting from the stress event. In some embodiments, a wearer's cortisol reactivity may be augmented by coordinated measurements of testosterone.

In the absence of continual sweat stimulation and a small sampling interval, which may not be sustainable or preferred for some applications, the sweat sensing device may need to determine that a stress event is in progress, for example by means of other sweat sensing device readings, GSR, a heart rate monitor, or other means. Once the device determines that a stress event is in progress, it can stimulate sweat or activate sensors to take the required measurements. In practice, the wearer's cortisol response as it manifests in sweat will lag the actual event, providing some flexibility in the lead time necessary for the device to take relevant measurements.

The sweat sensing device may use a cortisol reactivity reading by itself to estimate a propensity for risk-taking behavior. For example, a wearer with a large cortisol reactivity would tend to behave in a more risk averse manner, while a wearer displaying a muted cortisol reactivity would tend toward greater risk-taking behavior. The device's ability to translate cortisol reactivity into a risk-taking propensity may be improved by incorporating assessments of the basal cortisol, basal testosterone, and cortisol awakening response as discussed above. For example, an individual displaying exposure to chronic stress through high basal cortisol and muted CAR, when combined with a relatively small cortisol reactivity will have an increased propensity for risk-taking behavior. On the other hand, an individual displaying chronic stress exposure who subsequently registers a large cortisol reactivity in response to an event, may not have increased propensity for risk. As another example, a wearer indicating no chronic stress via basal cortisol and CAR levels, but having high basal testosterone, may have a muted cortisol reactivity, indicating an increased propensity for risk-taking behavior. As illustrated, cortisol reactivity may be used in conjunction with basal cortisol, basal testosterone, and CAR to improve assessment of risk-taking propensity.

Further, in addition to generalizations about wearers' reaction to chronic and short-term stress, aggregated data on individuals or similar phenotype individuals may be used to improve a device's ability to predict risk-taking propensity. For example, historical sweat sensor data on an individual or individuals as they react to acute stress may be used to construct an individual or phenotypical profile that will allow improved prediction of risk-taking behavior. Other relevant inputs may also improve the device's ability to predict risk-taking behavior, for example, information relevant to the wearer's dehydration level, blood sugar, fatigue level, or use of caffeine, alcohol or other substances.

In a preferred embodiment of the disclosure, the sweat sensing device may be configured to improve outcomes for financial traders. The device would accordingly be used to monitor a wearer as he or she engages in financial trading activity. By assessing the individual's basal cortisol, CAR, and in some cases, basal testosterone, the device could develop a general propensity for the individual to make riskier trading decisions. During trading activity, the device may also assess the individual's cortisol reactivity in response to short-term stress. If a wearer displayed propensity for risk over a certain threshold, an alert message may be sent to the wearer or device user. The device may also correlate trading frequency with cortisol levels to assess propensity for risk.

In some embodiments, the device may use other inputs to augment the device's ability to improve financial trading results, for example, the device may consider cognitive impairment due to dehydration, fatigue, caffeine, alcohol use, etc. For example, the wearer may be prompted to answer questions about their caffeine consumption for the day, or the device may include a sensor capable of measuring caffeine in sweat and correlating those measurements to a caffeine consumption amount. By determining the amount of caffeine consumed by a wearer, the device may be able to improve its predictive performance for risk-taking behaviors, or it might be able to correlate changes in cortisol or testosterone to caffeine consumption.

This has been a description of the present invention along with a preferred method of practicing the present invention, however the invention itself should only be defined by the appended claims.

1. A method of using a biofluid sensing device that is configured to be worn on an individual's skin to determine the individual's cortisol awakening response, comprising:
determining a period during which biofluid analytes are measured, where the measurement period begins within 1 hour after the individual wakes from a period of sleep;
taking at least one sweat analyte measurement during the measurement period, where at least one analyte is cortisol; and
using the at least one measurement to develop cortisol awakening response value; and
communicating the cortisol awakening response value to a user.

2. The method of claim 1 where sweat is stimulated to supply at least one sweat sample to the device.

3. The method of claim 1 where the measurement period is one of the following: at least 45 minutes; at least 1 hour; at least 30 minutes; and at least 15 minutes.

4. The method of claim 1 where the individual's waking state is determined by at least one of the following means: setting a wake time in a timekeeping means; using a sleep monitor; using a wearable device; measuring heart rate; measuring body temperature; and measuring the individual's movement and posture.

5. The method of claim 1 where at least one measurement is of an initial cortisol value, at least one measurement is of a peak cortisol value, and at least one measurement is of a post-peak cortisol value.

6. The method of claim 2 where the chronologically assured sweat sampling rate is increased during the cortisol awakening response period.

7. The method of claim 1 where the cortisol awakening response is compared to a diurnal cortisol profile for the individual.

8. (canceled)

9. A method of using a biofluid sensing device that is configured to be worn on an individual's skin to determine the individual's diurnal cortisol profile, comprising:
taking at least one sweat analyte measurement after the individual goes to sleep;
determining a basal cortisol level based on the at least one measurement taken while the individual's is asleep;
taking at least one sweat analyte measurement after the individual wakes; and
determining a peak cortisol level based on the at least one measurement taken while the individual is awake; and
using the basal cortisol level and peak cortisol level to develop the diurnal cortisol profile.

10. The method of claim 9 where the device takes at least one sweat analyte measurement at a time that is after the peak cortisol measurement.

11. The method of claim 9 where sweat is stimulated to provide at least one sweat sample to the device.

12. The method of claim 9 where the device determines that the individual has entered a sleep state by at least one of the following means: activation upon patch application; measuring heart rate, measuring body temperature; using a wearable device; using a sleep monitor; and measuring the individual's movement and posture.

13. The method of claim 9 where the individual's waking state is determined by at least one of the following means: setting a wake time in a timekeeping means; using a sleep monitor; using a wearable device; measuring heart rate; measuring body temperature; and measuring the individual's movement and posture.

14. The method of claim 9 where the diurnal cortisol profile is compared to a baseline cortisol profile.

15. (canceled)

16. The method of claim 11 where the chronologically assured sweat sampling rate is increased to measure the basal cortisol level.

17. The method of claim 11 where the chronologically assured sweat sampling rate is increased to measure the peak cortisol level.

18. The method of claim 11 where the chronologically assured sweat sampling rate is increased to measure the post-peak cortisol level.

19. The method of claim 11 where the chronologically assured sweat sampling rate is increased in response to at least one of the following factors: heart rate change; body temperature change; sweat rate change; sweat onset change; consumption of a meal; and occurrence of a stress event.

20. A method of determining an individual's stress level, comprising:
taking at least one sweat analyte measurement with a sweat sensing device, where at least one analyte is cortisol;
developing a stress profile value for the individual; and
communicating said value to a user.

21. (canceled)

22. The method of claim 20 where the analytes include at least two of the following: cortisol, Na+, Cl—, K+, glucose, adrenocorticotropic hormone, norepinephrine, epinephrine, dopamine, serotonin estrogen, and testosterone.

23. The method of claim 20 where the sweat sensing device measures at least one of sweat rate, sweat onset rate, sweat pH, and sensor temperature.

24. The method of claim 20 where the sweat sensing device is used to determine whether the individual has experienced one of the following conditions: a panic attack; a minor stress event; a chronic stress condition.

25. (canceled)

26. (canceled)

27. (canceled)

28. The method of claim 20 where the data includes at least one of the following: cortisol awakening response; diurnal cortisol; heart rate, and individual stress history.

29. A method of determining an individual's propensity for risk-taking behavior, comprising:
placing a sweat sensing device on the individual's skin;
using the device to determine a cortisol awakening response for the individual;
determining if the individual is subject to a chronic stress level; and
relating individual's chronic stress level to propensity for risk-taking behavior.

30. The method of claim 29 further comprising using the device to determine one of the following: a basal cortisol level for the individual; and a basal testosterone level for the individual.

31. (canceled)

32. The method of claim 29 further comprising using the device to determine a cortisol reactivity value for the individual, where the device takes at least one sweat cortisol measurement before the individual experiences a stress event; and the device takes at least one sweat cortisol measurement after the stress event.

33. The method of claim 29 where the method further includes using data relevant to at least one of the following to determine propensity for risk-taking behavior: the individual's hydration level; the individual's fatigue level; the individual's sleep history; the individual's cognitive function; the individual's caffeine consumption; the individual's nicotine consumption; and the individual's alcohol consumption.

34. A method of improving a performance metric for a financial trader, comprising:
using a sweat sensing device to determine the trader's propensity for risk-taking behavior; and
using the trader's risk-taking propensity to improve the performance metric.

35. The method of claim 34 where the metric is a ratio of financial gain to financial loss.

36. The method of claim 34 where the method also uses data relevant to at least one of the following to determine a risk-taking propensity: the individual's hydration level; the individual's sleep history; the individual's caffeine consumption; the individual's nicotine consumption; and the individual's alcohol consumption.

37. A device capable of determining sweat cortisol levels for an individual, comprising:
at least one primary sensor for measuring sweat cortisol;
at least one primary sensor for measuring a second sweat analyte;
at least one secondary sensor; and
a computation means.

38. The device of claim 37 where the device is configured to stimulate sweat in order to supply at least one sweat sample to the device.

39. (canceled)

40. The device of claim 37 where the device is configured to determine one of the following: a cortisol awakening response for the individual; and a diurnal cortisol profile for the individual.

41. (canceled)

42. The device of claim 37 where the device is configured to determine whether the individual has experienced one of the following: a panic attack; a minor stress event; a chronic stress condition.

43. (canceled)

44. (canceled)

45. The device of claim 46 where the device is configured to determine the individual's propensity for risk-taking behavior.

46. The device of claim 37, where at least one primary sensor is configured to measure sweat testosterone.
